(19) United States
(12) Patent Application Publication
(10) Pub. No.: US 2025/0215388 A1
(43) Pub. Date: Jul. 3, 2025

(54) HIGH pH METHODS AND COMPOSITIONS FOR CULTURING GENETICALLY MODIFIED HOST CELLS

(71) Applicant: AMYRIS, INC., Emeryville, CA (US)

(72) Inventors: Brandon FRIEDRIKSON, Emeryville, CA (US); Hanxiao JIANG, Emeryville, CA (US); Ninand D. KOTHARI, Emeryville, CA (US); Chi-Li LIU, Emeryville, CA (US); Adam MEADOWS, Emeryville, CA (US); Dominic PINEL, Emeryville, CA (US); Douglas J. PITERA, Emeryville, CA (US); Jessica WALTER, Emeryville, CA (US)

(21) Appl. No.: 18/869,250

(22) PCT Filed: Jun. 16, 2023
(86) PCT No.: PCT/US2023/025628

§ 371 Date: Nov. 25, 2024

Related U.S. Application Data
(60) Provisional application No. 63/352,898, filed on Jun. 16, 2022.

(51) Int. Cl. C12N 1/18; C12P 19/02; C12P 19/04; C12P 25/00

(52) U.S. Cl. CPC C12N 1/18; C12P 19/02; C12P 19/04; C12P 25/00

(57) ABSTRACT

The present disclosure relates to the methods and compositions for enhancing productivity or yield in fermentation. The methods and compositions provide culturing host cells at a high pH and compositions for culturing host cells at a high pH.

### TECHNICAL FIELD

The present disclosure relates to the use of high pH cell culture conditions to enhance cell growth and/or production of heterologous non-catabolic compounds by genetically modified host cells.

### BACKGROUND OF THE INVENTION

The advent of synthetic biology has brought about the promise of fermentative microbial production of biofuels, chemicals and biomaterials from renewable sources at industrial scale and quality. For example, functional non-native biological pathways have been successfully constructed in microbial hosts for the production of precursors to the antimalarial drug artemisinin (see, e.g., Martin et al., Nat Biotechnol 21:796-802 (2003); fatty acid derived fuels and chemicals (e.g., fatty esters, fatty alcohols and waxes; see, e.g., Steen et al., 2010, Nature 463:559-562); polyketide synthases that make cholesterol lowering drugs (see, e.g., Ma et al., 2009, Science 326:589-592); polyketides (see, e.g., Kodumal, 2004, Proc Natl Acad Sci USA 101:15573-15578); riboflavin (see, e.g., Kirby et al., 2015, Appl. Environ. Mircobiol. 81(1):130-138); and 2′-fucosyllactose (U.S. Pat. No. 10,519,475 B1). However, the commercial success of synthetic biology will depend largely on whether the production cost of renewable products can be made to compete with, or out-compete, the production costs of their respective non-renewable counterparts

Some of the largest costs of synthetic biology occur during fermentation. Much of these costs go toward the ingredients of the fermentation media including carbon sources, nitrogen sources, water, salts, and nutrients. Methods and compositions that improve the yields of fermentations will reduce the overall costs, making the production of renewable compounds more efficient and competitive.

### SUMMARY OF THE INVENTION

Provided herein are processes for culturing microbial cells in a fermentation medium. In certain embodiments, the methods comprise the steps of providing a population of microbial cells; and culturing the population in a culture medium at a pH selected from 5.1 to 7.0. In certain embodiments, the microbial cells are genetically modified host cells. In certain embodiments, the microbial cells produce one or more heterologous non-catabolic compounds. In certain embodiments, the microbial cells produce 2′-fucosyllactose. In certain embodiments, the microbial cells produce riboflavin. Advantageously, in particular embodiments, the methods can improve productivity or yield, or both, compared to culture methods with the same cells at a conventional pH, for instance pH 5.0. These method steps can be carried out with techniques and components apparent to those of skill in the art. Particular techniques and components are described in detail herein.

As described in detail below, the methods and compositions provided herein can increase productivity of a microbial strain by up to 15%, or more. As described in detail below, the methods and compositions provided herein can increase yield of a microbial strain by up to 15%, or more. An increase of 15% in productivity or yield provides a direct improvement in costs and efficiency for such a fermentation.

### BRIEF DESCRIPTION OF FIGURES

FIG. 1 provides normalized 2′-FL yield on sucrose (%; FIG. 1A, top left panel), normalized 2′-FL productivity (g/L/h; FIG. 1B, top right panel), normalized 2′-FL titer (g/kg; FIG. 1C, bottom left panel), and normalized accumulated lactose (g/kg; FIG. 1D, bottom right panel) at pH 5.0 and pH 5.5 for strain Y71081 and strain Y73923. All fermentation runs were at n=2 replication except Y73923 at pH 5.5, which was run at n=1.

FIG. 2 provides normalized 2′-FL yield on sucrose (%; FIG. 2A, top left panel), normalized 2′-FL productivity (g/L/h; FIG. 2B, top right panel), normalized average cell density (gDCW/L; FIG. 2C, bottom left panel), and normalized estimated maintenance (gTRS/gDCW/h; FIG. 2D, bottom right panel) at pH 5.0 and pH 5.5 for strain Y73923 at pH 5.0, pH 5.5, and pH 6.0. All fermentation runs were at n=2 replication except Y73923 at pH 5.5, which was run at n=1. DCW indicates dry cell weight.

FIG. 3 provides the percent TRS consumed out of total TRS fed for 2′-FL production, biomass production, or other, for Y73923 fermentations at pH 5.0 (n=2), pH 5.5 (n=1), and pH 6.0 (n=2). TRS indicates total reducing sugar.

FIG. 4 provides riboflavin yield relative to pH 5.0 at Day 6 (FIG. 4A, left panel) and productivity relative to pH 5.0 at Day 6 (FIG. 4B, right panel) for strain Y71840.

FIG. 5 provides riboflavin yield relative to pH 5.0 at Day 6 (FIG. 5A, left panel) and productivity relative to pH 5.0 at Day 6 (FIG. 5B, right panel) for strain Y76036.

### DESCRIPTION OF EMBODIMENTS

### 5.1 Definitions

As used herein, the term “genetically modified” refers to a host cell that comprises a heterologous nucleotide sequence.

As used herein, the term “heterologous” refers to what is not normally found in nature. For example, the term “heterologous” when used with respect to a nucleic acid (DNA or RNA) or protein refers to a nucleic acid or protein that does not occur naturally as part of the organism, cell, genome, or DNA or RNA sequence in which it is present, or that is found in a cell or location or locations in the genome or DNA or RNA sequence that differ from that in which it is found in nature. The term “heterologous” when used with respect to a nucleic acid (DNA) can also refer to a nucleic acid which is operably linked to a promoter other than an endogenous promoter. The term “heterologous compound” refers to the production of a compound by a cell that does not normally produce the compound, or to the production of a compound at a level at which it is not normally produced by the cell.

As used herein, the phrase “heterologous enzyme” refers to an enzyme that is not normally found in a given cell in nature. The term encompasses an enzyme that is:
(a) exogenous to a given cell (i.e., encoded by a nucleotide sequence that is not naturally present in the host cell or not naturally present in a given context in the host cell); and
(b) naturally found in the host cell (e.g., the enzyme is encoded by a nucleotide sequence that is endogenous to the cell) but that is produced in an unnatural amount (e.g., greater or lesser than that naturally found) in the host cell.

As used herein, the term “naturally occurring” refers to what is found in nature. For example, a maltose binding protein that is present in an organism can be isolated from a source in nature and that has not been intentionally modified by a human in the laboratory is a naturally occurring maltose binding protein (e.g., maltose binding protein sequences in GenBank). Conversely, as used herein, the term “naturally not occurring” refers to what is not found in nature but created by human intervention.

As used herein, “bio-organic compound” or “microbial-derived organic compound” is meant herein an organic compound that is made by microbial cells, including recombinant microbial cells as well as naturally occurring microbial cells.

The term “non-catabolic” refers to the process of constructing molecules from smaller units, and these reactions typically require energy. The term “non-catabolic compound” refers to a compound produced by a non-catabolic process.

The term “fermentation” is used to refer to culturing host cells that utilize carbon sources, such as sugar, as an energy source to produce a desired product.

The term “culture medium” refers to a medium which allows growth of cellular biomass and production of metabolites from host cells. It contains a source of carbon and may further contain a source of nitrogen, a source of phosphorus, a source of vitamins, a source of minerals, and the like.

As used herein, the term “fermentation medium” may be used synonymously with “culture medium.” Generally, the term “fermentation medium” may be used to refer to a medium which is suitable for culturing host cells for a prolonged time period to produce a desired compound.

The term “medium” refers to a culture medium and/or fermentation medium. The “medium” can be liquid or semi-solid. A given medium may be both a culture medium and a fermentation medium.

The term “whole cell broth” refers to the entire contents of a vessel (e.g., a flask, plate, fermentor and the like), including cells, aqueous phase, compounds produced in hydrocarbon phase and/or emulsion. Thus, the whole cell broth includes the mixture of a culture medium comprising water, carbon source (e.g., sugar), minerals, vitamins, other dissolved or suspended materials, microorganisms, metabolites and compounds produced by host cells, and all other constituents of the material held in the vessel in which a non-catabolic compound is being made by the host cells.

The term “fermentation composition” is used interchangeably with “whole cell broth.” The fermentation composition can also include an overlay if it is added to the vessel during fermentation.

As used herein, the term “production” generally refers to an amount of a compound produced by a host cell provided herein. In some embodiments, production is expressed as a yield of compound by the host cell. In other embodiments, production is expressed as a productivity of the host cell in producing the compound.

As used herein, the term “productivity” refers to production of a compound by a host cell, expressed as the amount of compound produced (by weight) per amount of fermentation broth in which the host cell is cultured (by volume) over time (per hour).

As used herein, the term “yield” refers to production of a compound by a host cell, expressed as the amount of compound produced per amount of carbon source consumed by the host cell, by weight.

### 5.2 Description

Provided herein are methods for culturing microbial cells at high pH. The microbial cells can be any microbial cells deemed useful to the person of skill in the art. In certain embodiments, the microbial cells are host cells. In certain embodiments, the microbial cells are genetically modified. In certain embodiments, the microbial cells are capable of producing a bio-organic compound. In certain embodiments, the host cells are capable of producing a heterologous compound. In certain embodiments, the host cells are capable of producing a heterologous, non-catabolic compound. In certain embodiments, the microbial cells are yeast cells. In certain embodiments, the microbial cells are S. cerevisiae cells. Useful microbial cells are described herein.

In certain embodiments, the microbial cells are capable of producing an isoprenoid. In certain embodiments, the microbial cells are capable of producing farnesane. In certain embodiments, the microbial cells are capable of producing a polyketide. In certain embodiments, the microbial cells are capable of producing a human milk oligosaccharide. In certain embodiments, the microbial cells are capable of producing 2′-fucosyllactose. In certain embodiments, the high pH of the fermentation composition limits cellular ATP demands needed to balance intracellular and extracellular pH. In certain embodiments, the microbial cells are capable of producing riboflavin. In certain embodiments, the high pH of the fermentation composition increases ammonium feeding to the cell facilitating the formation of a compound with one or more nitrogen atoms such as riboflavin.

The methods generally comprise the steps of: providing a population of microbial cells; and culturing the population in a culture medium at a pH selected from 5.1-7.0. In certain embodiments, the pH is selected from 5.3-6.0. In certain embodiments, the pH is about 5.3. In certain embodiments, the pH is about 5.5. In certain embodiments, the pH is about 6.0. With reference to a pH, in certain embodiments, the term “about” indicates a range of ±0.05. In certain embodiments, the pH is 5.3. In certain embodiments, the pH is 5.5. In certain embodiments, the pH is 6.0. In certain embodiments, the pH is measured to the indicated number of significant digits, as would be recognized by the person of skill. In certain embodiments, the pH is 5.30. In certain embodiments, the pH is 5.50. In certain embodiments, the pH is 6.00.

The pH can be maintained by standard techniques known to the person of skill. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In certain embodiments, the pH is maintained with hydrochloric acid and/or sodium hydroxide, as appropriate. The pH can be measured according to standard techniques known to the person of skill. In certain embodiments, the pH is measured with a conventional pH probe.

The fermentation composition can comprise any fermentation component deemed suitable to the person of skill without limitation. For instance, the fermentation composition can comprise ammonium phosphate, potassium phosphate, magnesium sulfate, trace elements, vitamins, trace metals, one or more carbon sources, and combinations thereof. Useful fermentation composition components are described herein.

In some embodiments, the methods described herein are carried out for a period of between 1 and 20 days. In some embodiments, the methods described herein are carried out for a period of between 1 and 10 days. In some embodiments, the methods described herein are carried out for a period of 1, 2, 3, 4, 5, 6, 7, 8, 9, 10, 11, 12, 13, 14, 15, 16, 17, 18, 19, 20 or more than 20 days. In some embodiments, the methods described herein are carried out for at least 1-10, 2-9, 3-7, or 4-6 days. In some embodiments, the methods described herein are carried out for 1-10, 2-9, 3-7, or 4-6 days.

The methods provided herein have provided improvements in productivity and yield for fermentations of microbial cultures, for instance microbial cultures producing a compound of interest. In certain embodiments, productivity is increased 1%, 2%, 3%, 4%, 5%, 6%, 7%, 8%, 9%, 10%, 15%, 20%, 25%, 30%, 35%, 40%, 45%, 50%, or more. In certain embodiments, yield is increased 5%, 10%, 15%, 20%, 25%, 30%, 35%, 40%, 45%, 50%, or more. In certain embodiments, productivity and yield are increased. In certain embodiments, biomass is increased 1%, 2%, 3%, 4%, 5%, 6%, 7%, 8%, 9%, 10%, 15%, 20%, 25%, 30%, 35%, 40%, 45%, 50%, or more. In certain embodiments, cell density is increased 1%, 2%, 3%, 4%, 5%, 6%, 7%, 8%, 9%, 10%, 15%, 20%, 25%, 30%, 35%, 40%, 45%, 50%, or more. In certain embodiments, host cell health is increased 1%, 2%, 3%, 4%, 5%, 6%, 7%, 8%, 9%, 10%, 15%, 20%, 25%, 30%, 35%, 40%, 45%, 50%, or more. In certain embodiments, ATP consumption is decreased 1%, 2%, 3%, 4%, 5%, 6%, 7%, 8%, 9%, 10%, 15%, 20%, 25%, 30%, 35%, 40%, 45%, 50%, or more. In these embodiments, changes are relative to the same cell strain grown under conventional conditions, i.e. without the processes described herein. In some embodiments, changes are relative to the same cell strain grown at pH 5.0 under otherwise comparable conditions.

### 5.3 Cell Cultures, Media, and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the cell culture, the fermentation, and the process.

The methods provided herein may be performed in a suitable culture medium (e.g., with or without pantothenate supplementation) in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a cell culture can subsist, i.e., maintain growth and viability. In some embodiments, the culture medium is an aqueous medium comprising assimilable carbon, nitrogen and phosphate sources. Such a medium can also include appropriate salts, minerals, metals and other nutrients.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

The concentration of a carbon source, such as glucose, in the culture medium should promote cell growth, but not be so high as to repress growth of the microorganism used. In preferred embodiments, the carbon source is at undetectable levels in the fermentation medium (e.g. at less than about 0.1 g/L). In such embodiments, the culture is carbon-limited, and culture cells should consume the carbon source as soon as it is delivered. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of isoprenoid. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As described elsewhere, the feed rate of the carbon source is adjusted according to the methods provided herein. The use of aliquots of the original culture medium may be desirable because the concentrations of certain nutrients in the medium (e.g. the nitrogen and phosphate sources) can be maintained simultaneously. Likewise, the trace metals concentrations can be maintained in the culture medium by addition of aliquots of the trace metals solution.

### 5.4 Microbial cells

The cell cultures can comprise any cells deemed useful by those of skill. Microbial cells useful in the compositions and methods provided herein include archae, prokaryotic, or eukaryotic cells. In certain embodiments, the microbial cells are recombinant, comprising one or more heterologous nucleic acids. In certain embodiments, the microbial cells are host cells, comprising one or more heterologous nucleic acids encoding one or more enzymes capable of catalysing the production of a compound of interest.

Suitable prokaryotic cells include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the cell is an Escherichia coli cell.

Suitable archaea cells include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic cells include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hase gawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the cell is a strain of the genus Candida, such as Candida lipolytica, Candida guilliermondii, Candida krusei, Candida pseudotropicalis, or Candida utilis.

In a particular embodiment, the cell is Saccharomyces cerevisiae. In some embodiments, the cell is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the cell is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the cell is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

In some embodiments, the cell is engineered to produce a C₅ isoprenoid. These compounds are derived from one isoprene unit and are also called hemiterpenes. An illustrative example of a hemiterpene is isoprene. In other embodiments, the isoprenoid is a C₁₀ isoprenoid. These compounds are derived from two isoprene units and are also called monoterpenes. Illustrative examples of monoterpenes are limonene, citranellol, geraniol, menthol, perillyl alcohol, linalool, thujone, and myrcene. In other embodiments, the isoprenoid is a C₁₅ isoprenoid. These compounds are derived from three isoprene units and are also called sesquiterpenes. Illustrative examples of sesquiterpenes are periplanone B, gingkolide B, amorphadiene, artemisinin, artemisinic acid, valencene, nootkatone, epi-cedrol, epi-aristolochene, farnesol, gossypol, sanonin, periplanone, forskolin, and patchoulol (which is also known as patchouli alcohol). In other embodiments, the isoprenoid is a C₂₀ isoprenoid. These compounds are derived from four isoprene units and also called diterpenes. Illustrative examples of diterpenes are casbene, eleutherobin, paclitaxel, prostratin, pseudopterosin, and taxadiene. In yet other examples, the isoprenoid is a C₂₀₊ isoprenoid. These compounds are derived from more than four isoprene units and include: triterpenes (C₃₀ isoprenoid compounds derived from 6 isoprene units) such as arbrusideE, bruceantin, testosterone, progesterone, cortisone, digitoxin, and squalene; tetraterpenes (C₄₀ isoprenoid compounds derived from 8 isoprenoids) such as β-carotene; and polyterpenes (C₄₀₊ isoprenoid compounds derived from more than 8 isoprene units) such as polyisoprene. In some embodiments, the isoprenoid is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene. Isoprenoid compounds also include, but are not limited to, carotenoids (such as lycopene, α- and β-carotene, α- and β-cryptoxanthin, bixin, zeaxanthin, astaxanthin, and lutein), steroid compounds, and compounds that are composed of isoprenoids modified by other chemical groups, such as mixed terpene-alkaloids, and coenzyme Q-10.

In some embodiments, the cell is engineered to produce a polyketide. In certain embodiments, the polyketide is selected from the group consisting of a polyketide macrolide, antibiotic, antifungal, cytostatic, anticholesterolemic, antiparasitic, a coccidiostatic, animal growth promoter and insecticide.

In some embodiments, the cell is engineered to produce a fatty acid.

Useful cells are described in WO 2015/095804, WO 2015/020649, and WO 2014/144135, the contents of which are hereby incorporated by reference in their entireties.

In some embodiments, the cell is engineered to produce a human milk oligosaccharide. In certain embodiments, the human milk oligosaccharide is 2′-fucosyllactose. Useful cells are described in U.S. Pat. No. 10,519,475 B1, which is hereby incorporated by reference in its entirety. In certain embodiments, the cell is engineered to express a lactose transporter (a proton symporter) from Kluyveromyces lactis (Lac12). In such embodiments, the cell is capable of catalyzing the formation of 2′-fucosyllactose from lactose in the fermentation composition with the cell's fucosyltransferase enzyme.

In some embodiments, the cell is engineered to produce riboflavin. Useful cells are described in Kirby et al., 2015, Appl. Environ. Mircobiol. 81(1):130-138), which is hereby incorporated by reference in its entirety.

### 5.5 Recovery of Compounds

Once compound is produced by the cell culture, it may be recovered or isolated for subsequent use using any suitable separation and purification methods known in the art. In some embodiments, an organic phase comprising the compound is separated from the fermentation by centrifugation. In other embodiments, an organic phase comprising the compound separates from the fermentation spontaneously. In other embodiments, an organic phase comprising the isoprenoid is separated from the fermentation by adding a demulsifier and/or a nucleating agent into the fermentation reaction. Illustrative examples of demulsifiers include flocculants and coagulants. Illustrative examples of nucleating agents include droplets of the isoprenoid itself and organic solvents such as dodecane, isopropyl myristrate, and methyl oleate.

The compound produced in these cells may be present in the culture supernatant and/or associated with the cells. In embodiments where the compound is associated with the cells, the recovery of the isoprenoid may comprise a method of permeabilizing or lysing the cells. Alternatively or simultaneously, the compound in the culture medium can be recovered using a recovery process including, but not limited to, chromatography, extraction, solvent extraction, membrane separation, electrodialysis, reverse osmosis, distillation, chemical derivatization and crystallization.

In some embodiments, the compound is separated from other products that may be present in the organic phase. In some embodiments, separation is achieved using adsorption, distillation, gas-liquid extraction (stripping), liquid-liquid extraction (solvent extraction), ultrafiltration, and standard chromatographic techniques.

### EXAMPLES

### Example 1

The present example provides substantial yield and productivity increases for cultures of yeast strains modified to produce 2′-fucosyllactose.

A control strain (Y71081) and a more advanced strain (Y73923) were chosen to optimize pH setpoints in a fermenter for the production of 2′-fucosyllactose (2′-FL). Lactose was used as a substrate during fermentation for the production of 2′-FL. Lactose is not imported or consumed by wild-type Saccharomyces cerevisiae.

2′-FL strains were engineered to import lactose from the fermentation media by expressing a lactose transporter (a proton symporter) from Kluyveromyces lactis (Lac12). Once inside the yeast cell, lactose appears to only be acted upon by the fucosyltransferase enzyme, which attaches a fucose moiety to lactose, making 2′-FL.

2′-FL production and export requires three ATP molecules in addition to biomass production and glycolysis to: pump out proton imported via the lactose symporter, to activate GDP-mannose, and to export 2′-FL. High sugar consumption for cellular maintenance and low growth rates in the production phase, among other reasons, during 2′-FL fermentations have indicated the possibility of ATP limitation in 2′-FL strains. It was hypothesized that maintaining a higher pH fermentation broth would reduce the ATP demand required to balance intra- vs. extra-cellular pH, thereby freeing up additional ATP required for 2′-FL and/or biomass production.

### Methods

A 0.25-L volume bioreactor was used to test the impact of elevated pH setpoints on 2′-FL production. The fermentation media included 14 g/L NH₄H₂PO₄, 5 g/L KH₂PO₄, 6 g/L MgSO₄, 10 mL/L trace elements solution, and 6 mL/L vitamins solution. The trace metals stock solution included 80 mM EDTA, 11.5 g/L ZnSO₄·7H₂O, 0.64 g/L CuSO₄, 0.64 MnCL₂·4H₂O, 0.94 g/L CoCl₂·6H₂O, 0.96 g/L Na₂MoO₄·2H₂O, 5.6 g/L FeSO₄·7H₂O, 5.8 g/L CaCl₂·2H₂O, and NaOH and HCl to adjust pH to 4.0. The vitamins stock solution included 0.2 g/L Biotin, 0.8 g/L p-aminobenzoic acid, 4 g/L calcium pantothenate, 4 g/L nicotinic acid, 10 g/L myoinositol, 4 g/L thiamine HCl, 4 g/L pyridoxine HCl, and NaOH to adjust the final pH to 6.5. 50 g/L maltose was included in the batch media and 20 g/L sucrose was separately added at the start of the fermentation as a feed pulse.

The main fermenter was inoculated with an initial OD of approximately 3 (30% v/v inoculum) and an initial culture volume of 0.115 L. The flask culture used to inoculate the bioreactor was grown to an OD of 8-12. Sucrose, the carbon source, and lactose, the substrate, were fed separately to the bioreactor at a predefined feedrate ratio in the fed-batch phase of the fermentation. The fermentation temperature was maintained at 30° C., while the pH was maintained at either 5.0, 5.5, or 6.0. 20% (v/v) ammonium hydroxide was used to control pH in the bioreactor.

After all of the ethanol produced during the batch phase of the fermentation by the culture was completely consumed, the first fed-batch phase was started initially with six 10 g TRS/L sucrose feed pulses with feed rates between 5 and 8 g TRS/L/h. Lactose feed delivery was started with the first fed-batch phase feed pulse with a lactose-H₂O:TRS feedrate ratio of 0.08. TRS indicates total reducing sugar. This feedrate ratio was then raised to 0.29 at the start of production fed-batch phase. The sucrose feedrate in the production phase was kept constant at 6 gTRS/L/h. At a lactose-H₂O:TRS feedrate ratio of 0.29, the feedrate of the production phase lactose feed pulses was 1.74 g lactose-H₂O/L/h. 24 g TRS/L (and therefore, 6.96 lactose-H₂O/L) were delivered in every pulse before the feed pulse was stopped to ensure complete consumption of carbon sources to prevent run-away ethanol or other metabolite formation.

The culture fermented for a total of 8 days with daily sampling and drawdown to 0.125 L. Media components other than the carbon source and substrate were added to bioreactor after every drawdown. The amount of post-sterile addition (PSA) media added was based on the size of the drawdown. The PSA media stock solution included 118.5 g/L NH₄H₂PO₄, 42.4 g/L KH₂PO₄, 50.8 g/L MgSO₄, 85 mL/L trace elements solution, and 51 mL/L vitamins solution. The components of the trace elements and vitamins stock solution are described above. The stir rate was automatically ramped from an initial set point of 1500 rpm in the batch phase to a constant stir rate of 2200 rpm as the dissolved O₂ decreased. During the production phase, the stir rate was maintained constant at 2200 rpm with a constant airflow rate of 250 mL/min. The culture was at micro-aerobic conditions for majority of the production phase with an oxygen uptake rate ranging from 80-130 mmol/L/h in the production phase. 2′-FL titer was measured every 24 hours and the amount of sucrose feed, lactose feed, media, and base added and the bioreactor vessel weight were monitored and recorded real-time with weigh scales throughout the fermentation process.

### Results

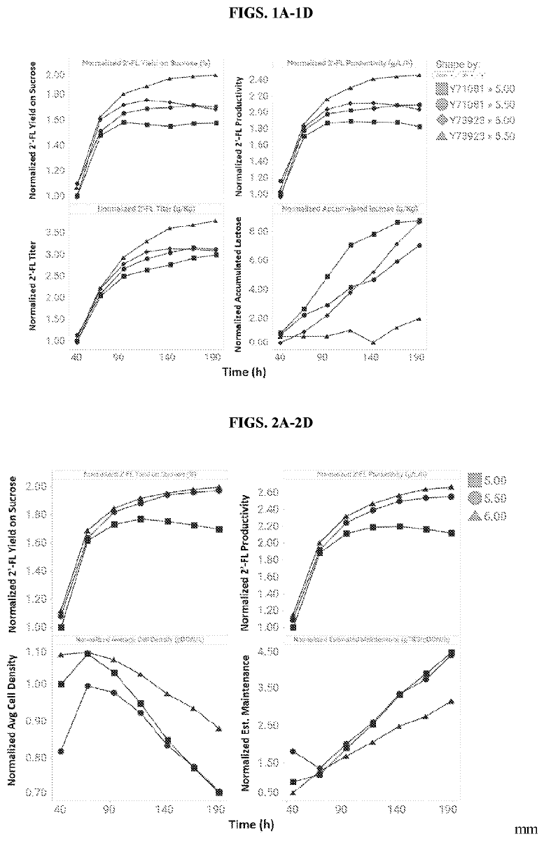

Y71081 (control) and Y73923 strains were fermented at pH 5.0 (control condition) and pH 5.5 to determine the impact of elevated pH setpoints on 2′-FL production performance. The cumulative 0-8-day 2′-FL yield on sucrose and productivity increased by approximately 16% and 21%, respectively, for strain Y73923 at a fermentation pH setpoint of 5.5 compared to the control pH condition of 5.0. Similarly, cumulative 0-8-day 2′-FL yield on sucrose and productivity were 8% and 15% higher for control strain Y71081 at pH setpoint of 5.5 compared to the control condition of pH 5.0. Out of all four conditions, almost complete lactose consumption was achieved at the elevated pH setpoint for the more advanced strain. FIG. 1 provides normalized 2′-FL yield (FIG. 1A), normalized 2′-FL productivity (FIG. 1B), normalized 2′-FL titer (FIG. 1C), and normalized accumulated lactose (FIG. 1D) at pH 5.0 and pH 5.5 for each strain.

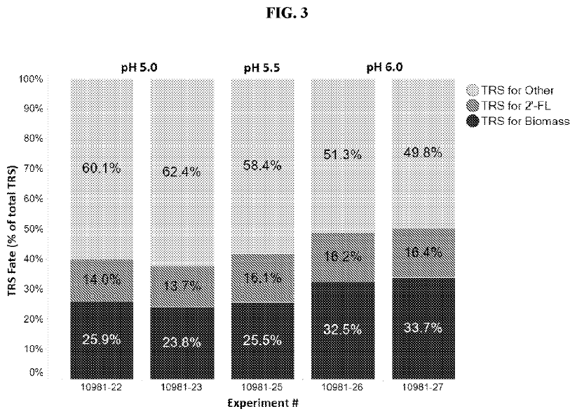

Strain Y73923 was also run in an additional condition of pH 6.0. While the 2′-FL production performance at pH 6.0 was improved over the performance observed at pH 5.5, the improvement was not as large. This was likely due to the limiting lactose substrate feed rates employed in these fermentation runs given that Y73923 was able to consume almost all of the lactose fed during the fermentation at both pH 5.5 and 6.0 setpoints. However, overall cell density and maintenance (estimated by calculating TRS consumed for purposes other than biomass or 2′-FL production) were dramatically improved at pH 6.0 compared to both pH 5.5 and pH 5.0. Overall, more TRS was consumed for 2′-FL and biomass production at pH 6.0 compared to pH 5.0, thereby reducing the cellular maintenance cost. FIG. 2 provides normalized 2′-FL yield (FIG. 2A), normalized 2′-FL productivity (FIG. 2B), normalized average cell density (FIG. 2C), and normalized estimated maintenance (FIG. 2D) at pH 5.0 and pH 5.5 for strain Y73923 at pH 5.0, pH 5.5, and pH 6.0. FIG. 3 provides the percent TRS consumed for 2′-FL production by strain Y73923 fermentations at pH 5.0 (n=2), pH 5.5 (n=1), and pH 6.0 (n=2).

While not intending to be bound by any particular theory of operation, higher 2′-FL and biomass production at elevated pH setpoints supports the ATP limitation hypothesis. The intracellular pH of Saccharomyces cerevisiae is around neutral (Orij, et al., 2012, Genome Biol 13, R80), higher than any of the external pH setpoints studied here. By increasing the external pH setpoint, the intra-vs. extra-cellular pH/proton gradient is reduced. In other words, the proton gradient against which the cells have to actively pump protons out of the cell in order to maintain its intracellular pH at optimum levels is reduced, thus potentially reducing the energy required to do so. Balancing the pH gradient may have, therefore, led to better cell health and production at elevated pH levels.

### Example 2

The present example provides substantial yield and productivity increases for cultures of yeast strains modified to produce riboflavin. It is reported in literature that increasing pH would increase yeast biomass and intracellular NH4⁺ concentrations. While not intending to be bound by any particular theory of operation, since each riboflavin molecule has four nitrogen atoms, it is hypothesized higher NH4⁺ will lead to higher riboflavin flux.

Riboflavin fermentations using yeast strain Y71840 were carried out in 0.5-L fermenters using sugar cane syrup as the feedstock. Each 0.5-L fermenter had initial volume of 0.25-L after inoculation. Initial fermenter temperature was controlled at 28° C. for 12-hr and then increased to 30° C. and maintained at 30° C. for the rest of 6-day fermentation process. pH was controlled at 5.00 (control, H11131-5&6), 5.30 (test, H11131-7&8) and 5.50 (test, H11131-9&10) by using NH₄OH solution.

Feeding strategy was based on delivering 110 mmol O₂/L/hr to the fermenter via airflow and agitation. After inoculation of the fermenter, a fill-and-draw process was executed about every 24-hr. This fill-and-draw process continued for 6-days until fermenters were harvested. The amount of sugar fed into the tank would allow the yeast cells to consume all delivered oxygen until the whole cell broth environment became microaerobic (when dissolved oxygen concentration reached zero).

Temperature (° C.), pH, feed rate (g/L/hr), tank weight (kg), feedstock weight, off-gas data such as CO₂, O₂ and ethanol were monitored and recorded automatically. pH was recalibrated at T=0 and T=48 with off-line pH probe to be within 0.05 pH unit. Daily whole cell broth sample was prepared for Riboflavin titer measurement and biomass measurement.

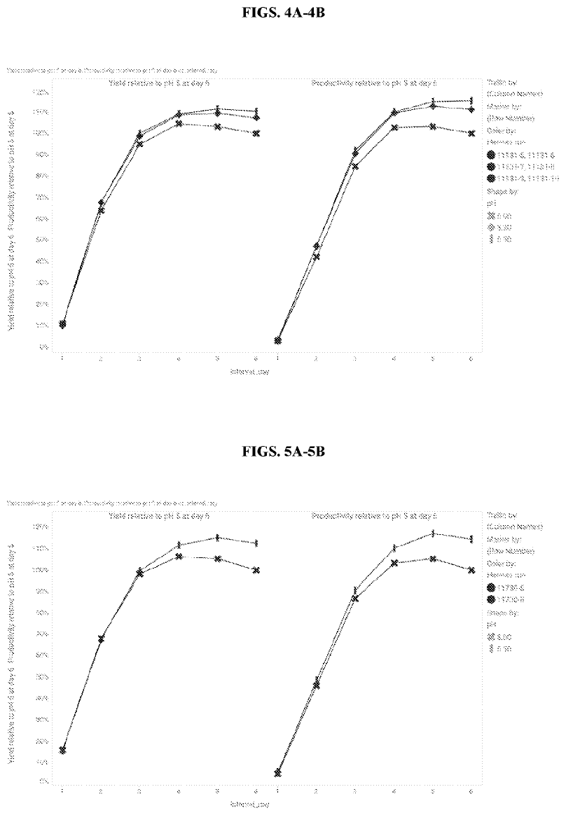

Yield and productivity for riboflavin fermentation in 0.5-L fermenter were improved when pH was controlled at 5.30 and 5.50 compared to pH 5.00. FIG. 4 provides yield relative to pH 5.0 at Day 6 (FIG. 4A) and productivity relative to pH 5.0 at Day 6 (FIG. 4B) for strain Y71840. Yield increased up to 10%, and productivity increased up to 15%.

### Example 3

The present example provides substantial yield and productivity increases for cultures of yeast strains modified to produce riboflavin. It is reported in literature that increasing pH would increase yeast biomass and intracellular NH4⁺ concentrations. While not intending to be bound by any particular theory of operation, since each riboflavin molecule has four nitrogen atoms, it is hypothesized that increasing pH will lead to higher riboflavin flux.

Riboflavin fermentations, in experiment H11730 and using yeast strain Y76036, were carried out in 2-L fermenters using sugar cane syrup as the feedstock. A 2-L fermenter had initial volume of 1.2-L after inoculation. Initial fermenter temperature was controlled at 28° C. for 12-hr and then increased to 30° C. and maintained at 30° C. for the rest of 6-day fermentation process. pH was controlled at 5.00 (control, H11730-5) and 5.50 (test, H11730-8) by using NH₄OH solution.

Feeding strategy was based on delivering 110 mmol O₂/L/hr to the fermenter. After inoculation of the fermenter, a fill-and-draw process was executed about every 24-hr. This fill-and-draw process continued for 6-day until fermenters were harvested. The amount of sugar fed into the tank would allow the yeast cells to consume all delivered oxygen until the whole cell broth environment became microaerobic (when dissolved oxygen concentration reached zero).

Temperature (° C.), pH, feed rate (g/L/hr), tank weight (kg), feedstock weight, off-gas data such as CO₂, O₂ and ethanol were monitored and recorded automatically. pH was recalibrated at T=0 and T=48 with off-line pH probe to be within 0.05 pH unit. Daily whole cell broth sample was prepared for riboflavin titer measurement and biomass measurement.

Yield and productivity for riboflavin fermentation in 2-L fermenter were improved when pH was controlled at 5.50 compared to pH 5.00. FIG. 5 provides yield relative to pH 5.0 at Day 6 (FIG. 5A) and productivity relative to pH 5.0 at Day 6 (FIG. 5B) for strain Y76036. Yield increased up to 13%, and productivity increased up to 14%.

The disclosure set forth above may encompass multiple distinct embodiments with independent utility. Although each of these embodiments has been disclosed in a certain form(s), the specific embodiments thereof as disclosed and illustrated herein are not to be considered in a limiting sense, because numerous variations are possible. The subject matter of this disclosure includes all novel and nonobvious combinations and subcombinations of the various elements, features, functions, and/or properties disclosed herein. The following claims particularly point out certain combinations and subcombinations regarded as novel and nonobvious. Embodiments in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of the present disclosure.

One or more features from any embodiments described herein or in the figures may be combined with one or more features of any other embodiments described herein or in the figures without departing from the scope of this disclosure.

All publications, patents, and patent applications cited in this specification are herein incorporated by reference as if each individual publication, patent, or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A method for culturing genetically modified host cells, comprising the steps of:
a. providing a population of genetically modified host cells; and
b. culturing the population in a culture medium at a pH selected from 5.1-7.0.

2. The method of claim 1, wherein the pH is selected from 5.3-6.0.

3. The method of claim 1, wherein the pH is about 5.3.

4. The method of claim 1, wherein the pH is about 5.5.

5. The method of claim 1, wherein the pH is about 6.0.

6. The method of any of the previous claims, wherein the genetically modified host cells are yeast cells.

7. The method of any of the previous claims, wherein the genetically modified host cells are S. cerevisiae cells.

8. The method of any of the previous claims, wherein the genetically modified host cells are capable of producing one or more heterologous non-catabolic compounds.

9. The method of claim 7, wherein the genetically modified host cells are capable of producing a human milk oligosaccharide.

10. The method of claim 8, wherein the genetically modified host cells are capable of producing 2′-fucosyllactose.

11. The method of any of the previous claims, wherein the genetically modified host cells are capable of producing a nitrogenous substance.

12. The method of claim 11, wherein the genetically modified host cells are capable of producing riboflavin.

13. The method of any of the previous claims wherein culturing at the pH is maintained for at least 1-10, 2-9, 3-7, or 4-6 days.

14. The method of any of the previous claims wherein culturing at the pH is maintained for 1-10 days.

15. The method of any of the previous claims wherein productivity is increased by at least 1%, 5%, 10%, 15%, or 20%, compared to the same cell strain grown under conventional conditions.

16. The method of any of the previous claims wherein yield is increased by at least 1%, 5%, 10%, 15%, or 20%, compared to the same cell strain grown under conventional conditions.

17. The method of any of the previous claims wherein biomass is increased by at least 1%, 5%, 10%, 15%, or 20%, compared to the same cell strain grown under conventional conditions.

18. The method of any of the previous claims wherein cell density is increased by at least 1%, 5%, 10%, 15%, or 20%, compared to the same cell strain grown under conventional conditions.

19. The method of any of the previous claims wherein host cell health is increased by at least 1%, 5%, 10%, 15%, or 20%, compared to the same cell strain grown under conventional conditions.

20. The method of any of the previous claims wherein ATP consumption is decreased by at least 1%, 5%, 10%, 15%, or 20%, compared to the same cell strain grown under conventional conditions.

21. A heterologous non-catabolic compound produced by the methods of any of the previous claims.

22. The heterologous non-catabolic compound of claim 21 that is a human milk oligosaccharide.

23. The heterologous non-catabolic compound of claim 21 that is 2′-fucosyllactose.

24. The heterologous non-catabolic compound of claim 21 that is a nitrogenous substance.

25. The heterologous non-catabolic compound of claim 21 that is riboflavin.
